(19) United States
(12) Patent Application Publication
(10) Pub. No.: US 2017/0367788 A1
(43) Pub. Date: Dec. 28, 2017

(54) Pressure Regulating Oral Appliance

(71) Applicant: Thomas Henry Collart, Endicott, NY (US)

(72) Inventor: Thomas Henry Collart, Endicott, NY (US)

(21) Appl. No.: 15/192,906

(22) Filed: Jun. 24, 2016

(51) Int. Cl. A61C 5/00; A61F 5/56

(52) U.S. Cl. CPC A61C 5/007; A61F 2005/563

(57) ABSTRACT

The Pressure Regulating Oral Appliance is an oral appliance which is designed to reduce reflux. The device prevents the buildup of negative pressure within the oral cavity that can create a vacuum promoting reflux of stomach content through the LES.

### CROSS-REFERENCE TO RELATED APPLICATIONS

The present application claims the benefit of provisional U.S. Application No. 62/185,174, filed Jun. 26, 2015, the full disclosure of which is incorporated herein by reference.

### STATEMENT REGARDING FEDERALLY SPONSORED RESEARCH OR DEVELOPMENT

(Not Applicable)

### REFERENCE TO SEQUENCE LISTING, A TABLE, OR A COMPUTER PROGRAM LISTING COMPACT DISC APPENDIX (IF APPLICABLE)

(Not Applicable)

### BACKGROUND OF THE INVENTION

This invention relates generally to medical devices and methods. In particular, the present invention relates to an oral device worn in the mouth of a patient to reduce the severity of reflux of stomach content into the esophagus.

The invention reduces reflux by preventing the buildup of negative pressure in the oral cavity and stimulating secretion of saliva. The contents of the stomach may be acidic and this acid as well as other stomach condense can be harmful to the tissue of the esophagus.

Persistent acid reflux (GERD—gastroesophageal reflux disease) is commonly treated by drug therapy aimed at reducing acid production, changes in eating habits and body elevation during sleep. In addition, surgery is sometimes used to help avoid acid reflux from the stomach into the esophagus through the Lower Esophageal Sphincter (LES).

Numerous devices exist which can affect the pressure in the oral cavity. Many of these address Obstructive Sleep Apnea (OSA), a serious medical condition resulting from a temporary airway blockage which occurs during sleep. These devices often support the technique of “continuous positive airway pressure” (CPAP). This technique maintains a continuous stream of pressurized air directly to the person's upper airway. Though this technique, incidentally, prevents the formation of negative pressure in the oral cavity it does so through an active device producing a pressurized airflow and can potentially result in pressure increase in the stomach. The present invention is a passive device and avoids the possibility of introducing pressurized air into the stomach.

### BRIEF SUMMARY OF THE INVENTION

The present invention, the Pressure Regulating Oral Appliance—hereinafter referred to as “the device”, eliminates formation of negative pressure within the oral cavity by allowing airflow into the mouth, thereby ameliorating vacuum induced flow through the

### DETAILED DESCRIPTION OF THE INVENTION

In the more elaborate of the 2 implementations illustrated, FIG. 2, a typical mouth guard structure provides overall support for the mechanism (10, 20). The bite surface (20) separates the upper and lower teeth and provides cushioning. The facing (10) fits between the teeth and lips to keep the device in place. The upper facing includes a contoured section (18) to fit comfortably around the superior labial frenulum. Unlike the implementation of FIG. 1, this device allows airflow even in individuals where separations around the teeth and gums are insufficient to allow airflow.

An external air path (12) provides a conduit for air flow into and out of the mouth by extending beyond the lips. This extension prevents the lips from blocking the port (14) and restricting air flow. The air passage (12, 16) extends interiorly through the facing and bite surface far enough to extend over the tongue. This extension (16) is somewhat flexible to allow tongue movement, but prevents the tongue from blocking air flow.

When properly positioned in the mouth the Pressure Regulating Oral Appliance allows free air flow into and out of the mouth preventing formation of negative pressure that can vacuum highly acidic stomach content into the esophagus. This benefit is particularly important when the wearer in in the supine position, such as during sleep. The device allows breathing through either the mouth or the nose.

FIG. 2 also illustrates that the functional elements of the device essential for air flow, (10, 12, 14, 16) can be incorporated with the elements of oral devices designed to LES. In addition to its ability to maintain neutral pressure in the oral cavity, use of the device does have some effect on swallowing which may positively affect LES relaxation, but this impact is not well understood. Use of the device can also reduce acidity within the esophagus by stimulating saliva production.

### BRIEF DESCRIPTION OF DRAWINGS

FIG. 1 is a superior view of one implementation of the Pressure Regulating Oral Appliance. It includes a soft mouth insert, item 10, with an additional structure for air flow. Item 12 provides an air passage which projects outward so as to prevent blockage of air flow by the lips. Item 14 is a tubular passage through the device to allow airflow. Overall, the design provides an appliance which fits the mouth comfortably between the lips and teeth. The device maintains position while allowing free airflow into and out of the mouth around separations in the teeth. Sustained airflow prevents the development of a vacuum within the oral cavity.

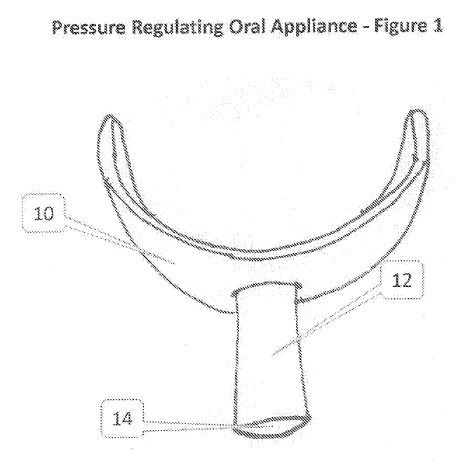

FIG. 2 is a superior view of one alternate implementation of the Pressure Regulating Oral Appliance. It includes a somewhat typical mouth guard structure, Item 10, with two additional elements. Item 12 provides an air passage which projects outward so as to prevent blockage of air flow by the lips. Item 16 serves to extend the air conduit back over the top of the tongue so that air flow is not blocked by the teeth or tongue. Overall, the design provides an appliance which fits the mouth comfortably and maintains position while allowing free airflow into and out of the mouth at all times. Sustained airflow prevents the development of a vacuum within the oral cavity.

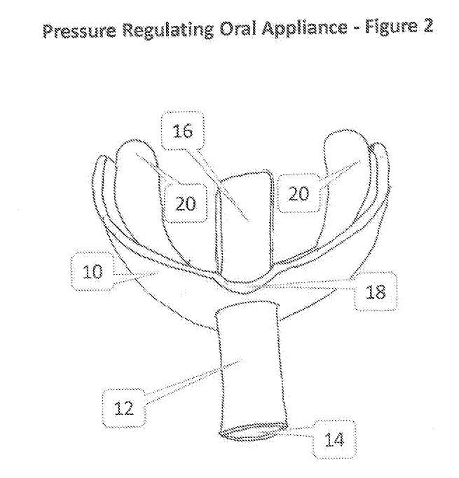

address other issues such as Temporomandibular Joint Disorders or Teeth Grinding (Bruxism)

1. The device described herein maintains airflow into and out of the mouth preventing the formation of negative pressure inside the oral cavity.

2. The device eliminates the formation of negative pressure within the oral cavity by allowing airflow into the mouth, thereby ameliorating vacume induced flow through the LES.

3. The functional elements of the device which provide air flow into the oral cavity can be incorporated into an oral device along with the elements designed to address other issues such as Temporomandibular Joint Disorders or Teeth Grinding (Bruxism)
